(19) United States
(12) Patent Application Publication
(10) Pub. No.: US 2006/0030625 A1
(43) Pub. Date: Feb. 9, 2006

(54) Dietary neurotransmitter precursors for balanced synthesis of neurotransmitters

(76) Inventors: Cheryle Ram Hart, Spokane, WA (US); Ronald Scott Grossman, Spokane, WA (US); Herbert Ram, Atlanta, GA (US)

Correspondence Address: ALAN J. HOWARTH, P.O. BOX 1909, SANDY, UT, 84091-1909, US

(21) Appl. No.: 10/913,027

(22) Filed: Aug. 6, 2004

(51) Int. Cl. A61K 31/198; A61K 31/137

(52) U.S. Cl. 514/567; 514/649

(57) ABSTRACT

Dietary supplements for treating a neurotransmitter deficiency include one or more precursors of the deficient neurotransmitter and a cofactor for activating in vivo enzymatic synthesis of the deficient neurotransmitter. The dietary supplements can also include an appropriate neurotransmitter, such as an amino acid. When administered through the oral mucosa, increases in levels of the deficient neurotransmitters and relief from deficiency symptoms are obtained.

### CROSS-REFERENCE TO RELATED APPLICATIONS

This application claims the benefit of U.S. Provisional Application No. 60/______ , filed Aug. 6, 2003, for Dietary Neurotransmitter Precursors for Balanced Synthesis of Neurotransmitters in a Slowly Dissolving Non-Glycemic Delivery System, which is hereby incorporated by reference in its entirety.

### STATEMENT REGARDING FEDERALLY SPONSORED RESEARCH OR DEVELOPMENT

Not applicable.

### BACKGROUND OF THE INVENTION

This invention relates to nutritional supplements. More particularly, this invention relates to nutritional supplements comprising precursors of neurotransmitters for increasing levels of neurotransmitters in the brain.

The brain is composed of billions of branching nerve cells called neurons. Neurons transmit messages from one cell to another, but never actually touch each other. Neurotransmitters are small messenger chemicals that are produced and stored in the nerve cell endings. When a neuron is activated, an electrical current passes through the cell to its branching nerve endings, causing the release of its neurotransmitters. Neurotransmitters diffuse into the space between cells (i.e., synaptic space). The neurotransmitters attach onto surfaces of neighboring neurons at special docking sites called receptors. When enough receptors are occupied, the cell is activated and an electrical current rapidly pulsates through the cell, causing release of its neurotransmitters. This electro-chemical process rapidly passes impulses through the chains of neurons, thus affecting millions of neurons in an instant.

The human brain makes over 100 different neurotransmitters—each programmed to relay special messages throughout the brain and body. Neurotransmitters are produced and stored in brain cells (neurons), and are released into action when neurons are electrically activated. Neurotransmitters are responsible for every thought, mood, pain, and pleasure sensation that humans feel. They control energy level, appetite, and food cravings, for example. Neurotransmitters regulate sleep and even sex drive. Two highly profiled neurotransmitters controlling mood, food, and energy are serotonin and dopamine. Norepinephrine is also recognized as an important neurotransmitter. Other well-studied neurotransmitters include glutamine, which stabilizes brain sugar levels and helps control against low blood sugar (hypoglycemia); endorphins, which are powerful mood boosters and pain relievers; and GABA (gamma-aminobutyric acid), which is a natural muscle relaxant.

Serotonin (5-hydroxytryptamine or 5-HT) is chemically classified as an indolamine and is known as a monoamine neurotransmitter. It was originally isolated from the blood serum as a substance causing powerful smooth muscle contraction. Only later was it demonstrated to be tryptamine with a hydroxyl group at the 5-position. Only 1-2% of the serotonin in the body is in the brain, insofar as serotonin is widely distributed in platelets, mast cells, and other cells. However, there is no equilibration between body serotonin and brain serotonin—the serotonin in the brain is independently synthesized from the amino acid tryptophan transported across the blood-brain barrier.

Serotonin synthesis is a 2-step process, the first step of which requires the enzyme tryptophan hydroxylase with oxygen, iron, and tetrahydrobiopterin (THB) as co-factors. Neither the enzyme nor the co-factors are rate-limiting for either step of these reactions—virtually all brain tryptophan is converted to serotonin. Serotonin concentration in the brain is far more sensitive to the effects of diet than any other monoamine neurotransmitter—and can be increased up to 10-fold by dietary supplementation in laboratory animals.

Consumption of a meal that is high in carbohydrate, branch-chained amino acids and tryptophan has a particularly dramatic effect because both glucose from carbohydrate and branch-chained amino acids (especially leucine) increase insulin secretion. Insulin facilitates the transport of the branch-chained amino acids into muscle cells, thereby reducing the competition tryptophan faces for the large neutral amino acid transporter that takes it across the blood-brain barrier. The resultant drowsiness induced by serotonin is a common effect of a large carbohydrate meal.

The richest concentration of serotonin in the body can be found in the pineal body, even though this gland does not use serotonin as a transmitter. Instead, serotonin is primarily used for synthesis of melatonin, so-called because it can darken the skin of amphibians (“melas” is Greek for “black”)—although it has also been reported to induce pigment lightening in cells. Melatonin is synthesized from serotonin in a 2-step process that takes an acetyl group from acetyl-CoA and a methyl group from SAM (S-adenosylmethionine).

Melatonin is of particular importance for regulating diurnal (circadian) and seasonal behavior and physiology in mammals. The pineal body has been called a “third eye” because its activity is influenced by light. In mammals, noradrenergic neurons near the optic nerve are inhibited by light. In darkness, norepinephrine stimulation of pineal cells causes the release of cyclic AMP second-messenger, which activates (phosphorylates) the N-acetyl transferase enzyme, which catalyzes acetylation of serotonin. Melatonin is a potent inhibitor of sexual activity in both sexes. Decreased melatonin in the spring leads to rutting in animals and the birth of offspring in the warmer seasons. Melatonin also stimulates production of brown adipose tissue, a special form of fat which (when burned) only produces heat, not ATP. This is especially important for hibernating animals.

Serotonin is responsible for feelings of well-being, serenity, mood stability and appetite satiety (fullness). Serotonin increases in the body when eating carbohydrates, especially sweet or starchy ones; crunching or chewing, such as “grazing” on carrots, sunflower seeds, or chewing gum; listening to soft music; meditating; taking baths; praying silently; slow dancing; laughing; using nicotine; eating “comfort” foods; drinking alcohol until feeling tired; doing yoga; getting a massage; crying; hugging, and caressing.

Serotonin seems to have distinctive actions contributing to anxiety and impulsive behavior. Patients with evidence of low serotonin levels have attempted suicide by very dramatic means. This may explain some of the therapeutic effects of fluoxetine (Prozac®), which selectively prevents the neuronal re-uptake of serotonin.

Monkeys with high levels of testosterone and low levels of serotonin are both aggressive and lacking in restraints on impulsive/violent behavior. Arsonists who commit their crime for mercenary reasons show normal levels of serotonin, but those who commit the crime impulsively have low serotonin. Lead interferes with serotonin synapse formation. Monkeys experimentally exposed to lead became so dangerously aggressive that the study was halted early, S. L. Wilkinson, A Recipe for Violence, 81 Chemical and Engineering News 33-37 (2003).

Dopamine and norepinephrine are also a monoamine neurotransmitters and are chemically classified as catecholeamines. Dopamine and norepinephrine are formed from the amino acids, phenylalanine and tyrosine. Tyrosine is produced in the liver from phenylalanine through the action of phenylalanine hydroxylase. However, tyrosine cannot be synthesized in the brain, and therefore must enter the brain by the large neutral amino acid transporter, which also transports phenylalanine, tryptophan, methionine, and the branch-chained amino acids. These amino acids all compete for the transporter, so a large quantity of one of the other amino acids in the blood stream could greatly limit the amount of tyrosine entering the brain. Once in the brain, tyrosine can be converted to dihydroxyphenylalanine (DOPA) by the tyrosine hydroxylase enzyme using oxygen, iron, and tetrahydrobiopterin (THB) as co-factors. High concentrations of dopamine inhibit tyrosine hydroxylase activity through an influence on the THB co-factor. DOPA is converted to dopamine by aromatic amino acid decarboxylase (which is fairly nonspecific insofar as it will decarboxylate any aromatic amino acid) using pyridoxal phosphate (PLP or vitamin B6) as a co-factor. This reaction is virtually instantaneous unless there is a vitamin B6 deficiency.

Dopamine and norepinephrine are primarily an inhibitory neurotransmitters that produce arousal. This may appear paradoxical, but the most likely explanation for this effect is that the postsynaptic cells for catecholamines themselves are inhibitory. There are 3-4 times more dopaminergic cells in the central nervous system (CNS) than adrenergic cells. There are two primary dopamine receptor-types: D1 (stimulatory) and D2 (inhibitory), both of which act through G-proteins. D2 receptors often occur on the dopaminergic neurons, partially for the purpose of providing negative feedback. These so-called autoreceptors can inhibit both dopamine synthesis and release.

Both dopamine and norepinephrine are catabolized by a two-step process involving the enzymes monoamineoxidase (MAO) and catechol-O-methyltransferase (COMT). COMT is primarily active in the synapses, and uses S-adenosyl methionine (SAM) as a methyl-group donor. MAO is primarily active in the pre-synaptic terminal against catecholamines that are not safely enclosed in storage vesicles. Normally, COMT only catabolizes about 10% of synaptic catecholamine, since catecholamine synaptic activity is primarily terminated by re-uptake into the pre-synaptic neuron terminal. MAO accounts for a much larger portion of catecholamine metabolism.

Dopamine is necessary for mental concentration, alertness, high energy, motivation, hunger regulation and sex drive. Dopamine increases when drinking or eating foods high in caffeine; listening and/or dancing to loud, fast music; taking showers; gospel singing; fast rhythmic dancing, like rock and roll; laughing; going on thrill rides; driving fast; smelling or seeing delectable foods; drinking alcohol and getting a “buzz;” and watching or participating in competitive sports.

When sufficient amounts of neurotransmitters are not available to dock onto receptors, the resulting brain electrical signal is weak. Under these circumstances, signs and symptoms of neurotransmitter deficiencies occur. Thus, proper amounts of neurotransmitters are necessary for maintaining optimal mental and physical health. Common conditions associated with serotonin/dopamine deficiencies include: depression, anxiety and panic attacks, chronic fatigue, fibromyalgia, headaches, especially migraines, premenstrual syndrome, appetite and eating disorders, especially binging or bulimia, seasonal affective disorder, addictions, attention deficit disorder, chronic pain, insomnia, irritability and anger disorders, low motivation, compulsive disorders, decreased sex desire.

Neurotransmitter levels can be measured by laboratory testing. Signs of deficiencies, however, can be easily recognized clinically by the symptoms they cause. A person's mood, behavior, attitude, energy level and certain thoughts toward food (i.e. cravings) give important clues about neurotransmitter levels. The types of food that are craved (e.g., starches, chocolate, or sweets) and times of day we crave them (late afternoon or evening) characterize specific neurotransmitter deficiencies.

Neurotransmitter deficiencies are caused by a variety of conditions or stimuli. For example, prolonged emotional or physical stress can lead to a neurotransmitter deficiency. The human body is programmed to handle sudden, acute or short bouts of stress. Prolonged, chronic stress takes it toll on the “fight or flight” stress hormones and neurotransmitters. Eventually, these become depleted and coping becomes more difficult.

Aging is another condition that can lead to neurotransmitter deficiency. Sixty percent of all adults past age 40 have some degree of neurotransmitter deficiency. Aging neurons make smaller amounts of neurotransmitters. Also, as people get older, the body does not respond as well to neurotransmitters.

Weight loss dieting can also lead to neurotransmitter deficiency. In fact, such dieting is the most common cause of self-induced neurotransmitter deficiencies. Limiting food intake to lose weight restricts the amounts of neurotransmitter precursors needed to produce enough neurotransmitters. Research has documented that women on diets significantly deplete their serotonin levels within three weeks of commencing dieting. This induced serotonin deficiency eventually leads to increased cravings, moodiness, and poor motivation. These all contribute to rebound weight gain—the most common, yet unfortunate, consequence of dieting.

Abnormal sleep is still another factor that can lead to neurotransmitter deficiency. Many neurotransmitters responsible for proper sleep, especially serotonin, are produced during rapid eye movement (REM) sleep in the early morning hours. Serotonin is converted to melatonin, the sleep hormone. When serotonin levels are low, melatonin levels will also be low. Disrupted sleep occurs and fewer neurotransmitters are produced, causing a vicious cycle of abnormal sleep.

Certain medications can also cause neurotransmitter deficiency. Long-term use of diet pills, stimulants, pain pills, narcotics and recreational drugs can deplete neurotransmitter stores. The use of ma huang, ephedra, and prescription diet pills (like phen-fen, Fastin®, and phentermine) use up large amounts of dopamine and serotonin. This can result in “rebound” appetite control problems, low energy, unstable mood, and sluggish metabolism.

Still further, neurotoxins can also cause neurotransmitter deficiency. Heavy metals, chemical pesticides, fertilizers, certain cleaning agents, industrial solvents, and recreational drugs cause damage to neurons and decrease neurotransmitter production. Excess caffeine, nicotine, and alcohol can be neurotoxic. The street drug, ecstasy (methylenedioxymethamphetamine or MDMA), has particularly concerning neurotoxic effects. Ecstasy can completely drain serotonin and permanently damage neurons, making treatment impossible. Ecstasy's chemical cousin, MDA, destroys cells that produce serotonin in the brain. Methamphetamine, also similar to ecstasy, damages brain cells that produce dopamine. Scientists have now shown that ecstacy not only makes the brain's nerve branches and endings degenerate, but also makes them “regrow, but abnormally—failing to reconnect with some brain areas and connecting elsewhere with the wrong areas. These reconnections may be permanent, resulting in cognitive impairments, changes in emotion, learning, memory, or hormone-like chemical abnormalities” (Delivering Results: A Program Report on Brain Research, Dana Alliance for Brain Initiatives, New York, 1996).

Hormone imbalances also cause neurotransmitter deficiency. Hormones influence neurotransmitter release and activity. If hormones are deficient or are off balance, neurotransmitters do not function well. Premenstrual syndrome (PMS) is a classic example of how low serotonin levels can temporarily shift each month. Mood, appetite, and sleep can be severely disrupted one to two weeks before the menstrual cycle. Another neurotransmitter imbalance occurs during menopause when dramatic changes in mood, energy, sleep, weight, and sexual desire occur.

Genetic predisposition also can lead to neurotransmitter deficiency. Some people are born with a limited ability to make adequate amounts of neurotransmitters. They exhibit deficiency symptoms as children or young adults and often have relatives who suffered from significant mental illnesses. As they get older, affected individuals experience even more profound symptoms and debilitation.

As recently as the 1970's the neuro-chemical pathways of the brain were not very well understood. There was very little in the way of successful treatments for mood disturbances. What medications did exist had many untoward and often serious side effects. Electroconvulsive or “shock” therapy (ECT) was about the only effective treatment for resistant severe depression. It was not then understood exactly how this therapy worked, but it is now realized that ECT works by artificially shocking neurotransmitters out of neurons. The resulting flood of neurotransmitters results in marked improvement of depression.

Advancements in neurochemistry in the 1980's fortunately lead to the discovery and understanding of many more neurotransmitters and their mechanisms of action. More options for treatment are now available, and researchers continue to develop even better ones. The most commonly prescribed medications for abnormal moods (dysphoria) are the serotonin re-uptake inhibitors, called SRI's. These include: Prozac®, Paxil®, Zoloft®, Effexor®, Serafem™, Serzone®, Celexa®, and Lexapro®. SRI's prevent serotonin from reabsorbing back into storage vesicles. More serotonin then stays in the synapse, reattaching to receptors and stimulating more neurons.

Many alternative methods aimed at raising neurotransmitter levels have been widely used with reported good success, especially in Asia and Europe. These methods include acupuncture, hypnosis, massage, reflexology, meditation, yoga, and herbal remedies. Neurotransmitter measurements of meditating Tibetan monks, showed increased levels of serotonin, the “serenity” messenger. With scientific data like these now supporting the benefits of these ancient treatments, more Western medical disciplines are becoming convinced and integrating them into their practices.

Neurotransmitter health must be maintained with a balanced diet that includes adequate amounts of protein, carbohydrates and fats. No food group can be eliminated, since they are all critical for proper neurotransmitter production and function. Dietary neurotoxins, like excess caffeine, nicotine and alcohol, decrease production and should be avoided.

Most neurotransmitters are made from protein or its subunits, amino acids. Eating adequate amounts of dietary protein is critical to avoiding neurotransmitter deficiencies. The average person requires 40-70 grams (up to 90 grams for a very active athlete) of protein daily. Serotonin is synthesized in the body from the amino acid, tryptophan. Tryptophan is the least common amino acid in food. It is also the most difficult amino acid to absorb into the brain. These make serotonin synthesis more difficult.

Although tryptophan is mainly found in fish, meat, dairy products, eggs, nuts, and wheat germ, eating these foods does not substantially increase serotonin. This is because these foods contain other amino acids that compete with tryptophan for absorption.

Surprisingly, eating carbohydrates raises serotonin levels, but eating protein decreases serotonin levels. Carbohydrates cause an insulin response that favors tryptophan absorption over other amino acids. This explains why many people who need more serotonin (like those who are over-stressed or depressed) start to “self-medicate” by eating more sweets or starchy carbohydrates. As tryptophan absorption increases after eating these foods, serotonin production also increases, and the person feels better.

Research has shown that women on high protein/very low carbohydrate diets lower their serotonin levels, making them more prone to weight gain relapse, depression, excessive craving, binging, bulimia, severe PMS, and seasonal affective disorder.

Dopamine is made from the amino acid tyrosine. Eating high protein foods promote dopamine production. Tyrosine is abundant and is found in chicken, fish, dairy products, almonds, avocados, bananas, legumes, soy products, pumpkin, and sesame seeds.

Dietary carbohydrates play a critical role in brain health. Women, especially, are vulnerable to how carbohydrates affect their moods. Serotonin, the main neurotransmitter for mood and appetite regulation, depends on carbohydrates for synthesis. Research results have linked serotonin deficiency conditions to low dietary carbohydrate intake. Women normally have one third less serotonin than men. Diets that severely restrict carbohydrates will result in even lower serotonin levels. Women on high protein/very low carbohydrate diets are at greater risk for depression, seasonal affective disorder (SAD), carbohydrate crave/binge disorder, and severe premenstrual syndrome.

About two thirds of our brain is made of fat (lipids). Lipids are incorporated into brain cell walls, promoting membrane flexibility and strength. A filmy fat layer covers the branches of neurons, allowing proper electrical transmission of brain signals. Most lipids can be made directly by the body, but two lipids, called essential fatty acids (EFA) can come only from food. The cell membranes of neurons are made from the essential fatty acids alpha-linoleic acid (ALA) and linoleic acid (LA). ALA belongs to the “omega-3” fatty acid family. Main food sources of omega-3 ALA include flax seeds, walnuts, sea plants, and green leafy vegetables. Linoleic acid (LA) belongs to the “omega-6” fatty acid family. LA is found in the oils of seeds and nuts. Main food sources of omega 6 LA include cold-pressed sunflower, safflower, corn and sesame oils.

The most abundant fat in the brain is DHA (docosahexaenoic acid), an omega-3 fatty acid. Good dietary sources of DHA come from high-fat, cold water fish like salmon, sardines, mackerel, and trout. DHA made from microalgae is also available in supplement capsules.

A balanced 1:1 ratio of omega-6 to omega-3 fatty acids is necessary for healthy brain development and function. Unfortunately, Western diets are overly concentrated in omega-6 fats (from meat and dairy) in a ratio of 20:1 or higher. This imbalance can lead to a variety of disorders, including hyperactivity, depression, schizophrenia, and other mental disorders. Infants deprived of adequate dietary fats or given improper fat ratios during development have smaller brains and lower I.Q. scores.

Correction of this ratio imbalance is encouraged by eating more omega-3-rich fish and flax seed oil and less omega-6 foods. Avoidance of all trans-fatty acids found in partially-hydrogenated oils, margarine, and shortenings is also recommended.

Nutrition experts agree that, as a minimum, at least 7% of daily calories should come from dietary fats. Weight-loss diets containing 20-30% percent of calories from dietary fat (27-60 grams of fat daily for women and 33-73 grams for most men) is the current recommendation made by health experts.

Individuals who crave fried or rich, buttery food, have dry skin, hair, and eyes, or chronic constipation often have deficiencies of omega-3 fatty acids. Omega-3 replacement by dietary means or supplements often eliminates these symptoms.

Dietary corrections are important for restoring healthy brain function, but may not be enough to correct a significant neurotransmitter deficiency. Foods vary in their concentrations of amino acids, and intestinal absorption can be unpredictable. The amount of protein needed to replace depleted neurotransmitters is not practical or healthy to consume. For example, one would have to eat a 32-ounce steak or 3-dozen eggs every day to keep up with the amount of amino acids needed to improve PMS symptoms caused by low neurotransmitter levels.

Practical ways to naturally increase neurotransmitters with dietary supplements are now being utilized. A new class of supplements, neuro-nutraceuticals, has shown promising results. This method provides the brain with adequate amounts of basic building blocks (neurotransmitter precursors) needed for neurotransmitter production. Recently published studies support the validity of using supplements to raise neurotransmitter levels. Laboratory measurements and clinical studies have documented predictable rises in neurotransmitter levels and symptom improvements.

In view of the foregoing, it will be appreciated that providing nutritional supplements comprising precursors of neurotransmitters for increasing levels of neurotransmitters in the brain would be a significant advancement in the art.

### BRIEF SUMMARY OF THE INVENTION

A dietary supplement composition for treating neurotransmitter deficiencies comprises a mixture of a first precursor of a first neurotransmitter and a cofactor for activating in vivo enzymatic synthesis of the first neurotransmitter from the first precursor or of a second neurotransmitter. An illustrative cofactor according to the present invention comprises vitamin B-6. Illustrative neurotransmitters include dopamine, norepinephrine, and serotonin, and illustrative precursors include DOPA, tyrosine, and 5-hydroxytryptophan. The dietary supplement composition can further comprise a neurotransmitter, such as an amino acid, such as glutamine. The dietary supplement composition can further comprise a low-digestible carbohydrate sweetener, such as isomalt, maltitol, and mixtures thereof. Still further, the composition can also contain a non-nutritive sweetener, such as acesulfame-K, sucralose, and mixtures thereof.

Another illustrative embodiment of the invention comprises a dietary supplement composition comprising a mixture of a first precursor of a first neurotransmitter and a cofactor for activating in vivo enzymatic synthesis of a second neurotransmitter.

Still another illustrative embodiment of the invention comprises a dietary supplement composition comprising a mixture of:
(a) a base comprising a low-digestible carbohydrate sweetener;
(b) a cofactor for activating in vivo enzymatic synthesis of a first neurotransmitter; and
(c) a precursor of the first neurotransmitter or of a second neurotransmitter.

Yet another illustrative embodiment of the invention comprises a method of treating a neurotransmitter deficiency comprising orally administering an effective amount of a dietary supplement composition comprising a mixture of a first precursor of a first neurotransmitter and a cofactor for activating in vivo enzymatic synthesis of the first neurotransmitter from the first precursor or of a second neurotransmitter. Illustratively, the dietary supplement composition is administered such that the first precursor and the cofactor are absorbed through the mucosa of the oral cavity.

### BRIEF DESCRIPTION OF THE SEVERAL VIEWS OF THE DRAWINGS

FIG. 1 shows a bar graph of the average percent change of dopamine after treatment according to the present invention as described in Example 1.

FIG. 2 shows a bar graph of the average percent change of serotonin after treatment according to the present invention as described in Example 1.

FIG. 3 shows a bar graph of the percent improvement in symptoms over time according to the present invention as described in Example 2.

FIG. 4 shows a bar graph of the percent improvement in appetite control according to the present invention as described in Example 3: active (open bars); placebo (shaded bars).

FIG. 5 shows a bar graph of the percent improvement in cravings symptoms according to the present invention as described in Example 3: active (open bars); placebo (shaded bars).

FIG. 6 shows a bar graph of the percent improvement in emotional symptoms according to the present invention as described in Example 3: active (open bars); placebo (shaded bars).

FIG. 7 shows a bar graph of the percent improvement in mood according to the present invention as described in Example 3: active (open bars); placebo (shaded bars).

FIG. 8 shows a bar graph of the percent improvement in sleep and energy according to the present invention as described in Example 3: active (open bars); placebo (shaded bars).

FIG. 9 shows a bar graph of the percent improvement in muscle-related symptoms according to the present invention as described in Example 3: active (open bars); placebo (shaded bars).

### DETAILED DESCRIPTION

Before the present nutritional supplements and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a dietary supplement composition comprising “a precursor” includes reference to a mixture of two or more of such precursors, reference to “a cofactor” includes reference to one or more of such cofactors, and reference to “a neurotransmitter” includes reference to two or more of such neurotransmitters.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, “low-digestible carbohydrate sweetener” means a carbohydrate that is sweet to the taste yet contains very low caloric content compared to sucrose, because the low-digestible carbohydrate sweetener is digested by enzymes in the body at a low rate compared to that of sucrose. Isomalt is illustrative of such sweeteners. Isomalt is an approximately equimolar mixture of 6-O-α-D-glucopyranosido-D-sorbitol and 1-O-α-D-glucopyranosido-D-mannitol dihydrate. Isomalt is made from beet sugar. In a two step process, the sugar components glucose and fructose are used to make isomalt, which looks like sugar and tastes like sugar, but only has half the calories of sugar. The reason for the reduced calorie content is because human enzymes digest isomalt in much smaller amounts and more slowly than sugar. As a result, blood sugar and insulin levels do not change following its consumption. The human body uses about 50% of isomalt. “Low digestible carbohydrates” belong to a fiber group that stimulate bowel activity and help counteract constipation. Isomalt does not promote dental cavities and has a very low glycemic index. It is currently being used in some confectionery and food products as an alternative to conventional sugars and sweeteners. Another illustrative low-digestible carbohydrate sweetener is maltitol.

As used herein, “non-nutritive sweetener” means a sweetener that is essentially not digested in the body. Illustrative non-nutritive sweeteners include acesulfame-K, sucralose, and the like. Such non-nutritive sweeteners are well known in the art and are commercially available from a variety of sources.

As used herein, “effective amount” means an amount of a neurotransmitter precursor, neurotransmitter, vitamin, cofactor, or the like that is nontoxic but sufficient to provide the desired local or systemic effect and performance at a reasonable benefit/risk ratio attending any treatment of a deficiency condition by dietary supplementation. For example, an effective amount of a neurotransmitter precursor is an amount sufficient to achieve measurable relief from deficiency symptoms when administered at a nontoxic dose for a selected period of time.

As used herein, “flavoring agents” or “natural and artificial flavorings” and similar terms mean agents that are generally regarded as safe for using in flavoring foods and drugs. Such agents vary considerably in their chemical structure, ranging from simple esters, alcohols, and aldehydes to carbohydrates and complex volatile oils. Synthetic flavors of almost any desired type are now available.

As used herein, “vitamin B-6” does not denote a single substance, but is rather a collective term for a group of naturally occurring pyridines that are metabolically and functionally interrelated: namely, pyridoxine, pyridoxal, pyridoxal phosphate, and pyridoxamine. They are interconvertible in vivo in their phosphorylated form. Vitamin B-6 in the form of pyridoxal phosphate or pyridoxamine phosphate functions in carbohydrate, fat, and protein metabolism. Its major functions are most closely related to protein and amino acid metabolism. The vitamin is a part of the molecular configuration of many enzymes (a coenzyme), notably glycogen phosphorylase, various transaminases, decarboxylases, and deaminases. The latter three are essential for the anabolism and catabolism of proteins. Pyridoxine is also aids in fat and carbohydrate metabolism; aids in the formation of antibodies; maintains the central nervous system; aids in the removal of excess fluid of premenstrual women; promotes healthy skin; reduces muscle spasms, leg cramps, hand numbness, nausea and stiffness of hands; and helps maintain a proper balance of sodium and phosphorous in the body. Vitamin B-6 is also a cofactor for synthesis of dopamine from DOPA.

The dietary supplements according to the present invention can be formulated as “hard candy” lozenges, compressed lozenges, compressed gum-like substances, or other forms as are well known in the art. These dosage forms are primarily intended to be dissolved in the mouth such that the active ingredients are absorbed slowly by the blood vessels lining the mouth, whereby the active ingredients pass directly into the blood stream and can be translocated to the brain without having to pass through the digestive tract. Absorption of active ingredients through the oral mucosa (e.g., sublingual, buccal, and the like) is well known in the art. This route of delivery provides for direct, immediate delivery to the brain through the oral membranes, improves symptoms faster and at lower doses than pill form supplements. Delivery through the oral mucosa bypasses digestion, eliminating time constraints around meals. Mucosal absorption also minimizes stomach and intestinal upset commonly experienced with capsule or tablet forms.

The use of low-digestible carbohydrate sweeteners as a base for carrying the active ingredients is advantageous because it avoids adding sugar to the diet, does not promote dental caries, and is safe for use by diabetics.

The ingredients are mixed together in the selected weight ratios. Illustrative weight ratios for a composition comprising tyrosine, DOPA, L-glutamine, and L-5-hydroxytryptophan range from about 1:0.5:0:0.5 to about 1:5:5:10. Ingredients can be USP or equivalent pharmaceutical grade, food grade formulations, or can be extracts of natural ingredients. For example, DOPA can be substituted by velvet bean (Mucuna pruriens seed extract. Velvet bean seeds contain about 5% by weight of DOPA. Similarly, L-5-hydroxytryptophan can be substituted by Griffonia simpilicifolia seed extract, which is high in 5-HTP.

An illustrative method of making a “hard candy” lozenge as known in the art will now be described. Hard candies are basically very high dry substance liquids, consisting of sweeteners (traditionally sugar and glucose syrup), which are cooked to remove the majority of their water and then cooled to form a stable “glass”. Acids, colors, and flavorings are added after this stage. In tradition sugar-containing hard candies, the sucrose is prevented from crystallizing on cooling by the addition of glucose syrup, known as “doctoring syrup”. Sugar-free hard candies are produced using a similar process except for the cooking temperature, which is higher, followed by a longer cooling time. Maltitol syrup, which can be used to replace both sugar and glucose, was the first product found to yield a candy glass that was sufficiently sweet, clear, and stable for sugar-free hard candies. The maltitol level and the hydrogenated oligosaccharides in the maltitol syrup control the crystallizing property of maltitol in the same way as glucose. However, the higher hygroscopicity of maltitol syrup requires the candy mass to be cooked to a very high dry substance of about 99% to obtain a candy with an acceptable shelf life. This is achieved by cooking the candy mass to 168° C. and applying 5 minutes of vacuum to reach a residual moisture of less than 1%. Maltitol syrup-based hard candies need to be wrapped and packed immediately after cooling in order to avoid moisture pick-up.

An illustrative recipe for making a sugar-free lozenge involves dissolving the sugar-free sweetener in water. The resulting solution is then heated to 155° C. and vacuum is applied for 5 minutes to obtain a mass with 1.5% moisture content. The mass is then cooled to 90° C. before adding active ingredients, sweeteners, and flavorings. The mass can be shaped by forming, depositing or plastic molding as required. The lozenges should be kept at low relative humidity, (50% RH) and at room temperature to prevent moisture pick-up before packaging (either unwrapped or individually wrapped.

Dosage forms according to the present invention generally comprises one or two lozenges per serving. One or two lozenges are dissolved in the mouth every three to four hours. Chewing or swallowing the lozenges whole should be avoided to obtain the desired effect.

### Example 1

An illustrative dietary supplement composition according to the present invention was configured to elevate serotonin and dopamine levels in a balanced manner. This composition was formulated as “hard candy” lozenges according to methods well known in the art. The ingredients were 2 g isomalt, 12 mg vitamin B-6 (as pyridoxine hydrochloride); and 390 mg of a blend of DOPA (as velvet bean (Mucuna pruriens seed extract), tyrosine (as L-tyrosine HCl), L-glutamine, and L-5-hydroxytryptophan (5-HTP; as Griffonia simplicifolia seed extract); and minor amounts of natural and artificial flavorings, acesulfame K, and sucralose. The tyrosine, DOPA, L-glutamine, and L-5-hydroxytryptophan were mixed in weight ratios ranging from 1:0.5:0:0.05 to 1:5:5:10.

### Example 2

An illustrative dietary supplement composition according to the present invention was configured to primarily elevate dopamine levels. This composition was formulated as “hard candy” lozenges according to methods well known in the art. The ingredients were 1 g isomalt, 6 mg vitamin B-6 (as pyridoxine hydrochloride); and 175 mg of a blend of DOPA (as velvet bean (Mucuna pruriens seed extract), tyrosine (as L-tyrosine HCl), and L-glutamine; and minor amounts of natural and artificial flavorings, acesulfame K, and sucralose. The tyrosine, DOPA, and L-glutamine were mixed in weight ratios ranging from 1:0.5:0.05 to 1:5:5.

### Example 3

An illustrative dietary supplement composition according to the present invention was configured to primarily elevate serotonin levels. This composition was formulated as “hard candy” lozenges according to methods well known in the art. The ingredients were 1 g isomalt, 6 mg vitamin B-6 (as pyridoxine hydrochloride); 20 mg of L-5-hydroxytryptophan (5-HTP; as Griffonia simplicifolia seed extract); and minor amounts of natural and artificial flavorings, acesulfame K, and sucralose.

### Example 4

Double-Blind Clinical Trial

Twenty participants were recruited and divided into two groups according to gender—11 women and 9 men. Each participant was arbitrarily assigned to receive either a product lozenge or a placebo lozenge. Each participant completed a medical history, current medication and dietary supplement form. Participants were instructed to avoid taking all medications, vitamins, or supplements on the day of the study until they had completed the study. No food was permitted and only water was allowed for consumption until completion of the study.

Each participant provided a pre-dose morning urine collection (second void of the morning). Then subjects orally dissolved their assigned testing lozenge, prepared according to Example 1 (placebo or active). Participants drank only water and ate no food until they completed their final post-dose urine collection over the next 2 hours.

All urine samples were labeled and frozen in lab-provided vials and submitted to Pharmasan Lab (Osceola, Wis.) by overnight delivery. All samples were submitted for measurement of dopamine and serotonin. Collection of urine specimens for study followed the protocol provided by the testing laboratory. Normal ranges and optimal ranges of excreted neurotransmitter levels were determined.

Baseline urinary excretion levels of the neurotransmitters reflect a central—peripheral equilibrium. This means that initial low levels of neurotransmitters measured in the urine (peripheral) directly reflect low reservoir levels of neurotransmitters in the brain (central). The lower the urinary amount, the more deficient a person is in brain neurotransmitters. With the addition of neurotransmitter enhancing compounds, referred to as neurotransmitter precursor therapy, levels of neurotransmitters measured in the urine rise. This rise also indicates increased central (brain) synthesis of neurotransmitters.

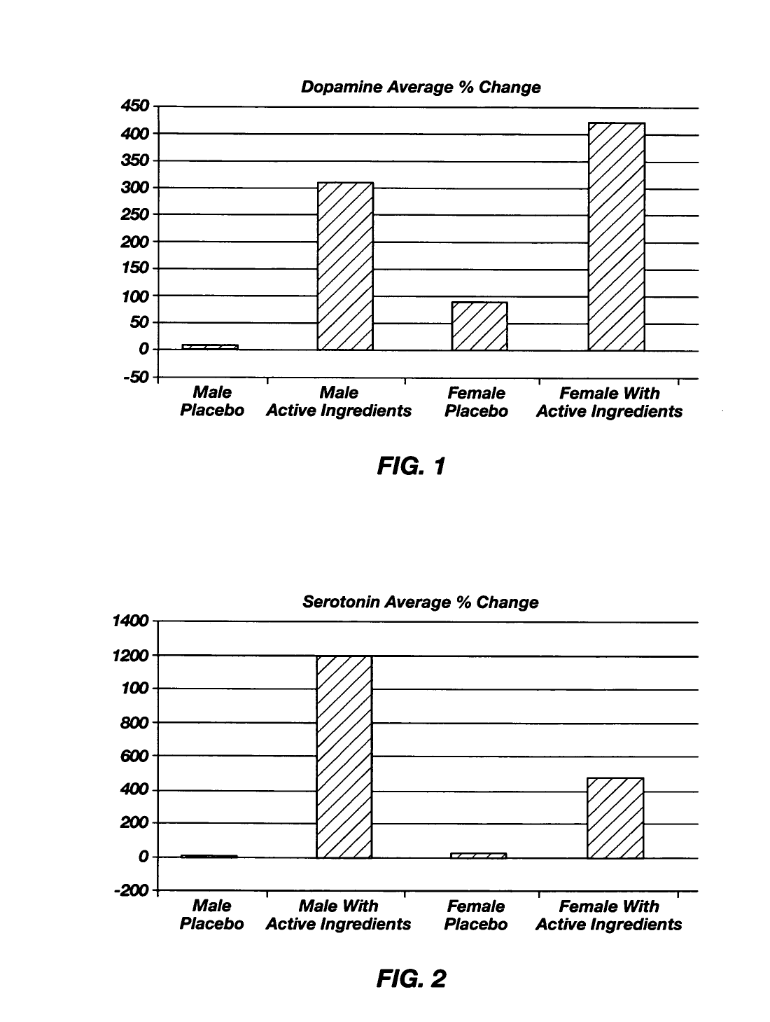

FIGS. 1 and 2 show that all placebo patients obtain ed results within a very narrow range, showing very little, if any effect. The group receiving the product lozenge (active ingredients) demonstrated an average 819% rise in both serotonin and 425% rise in dopamine levels.

### Example 5

Clinical Symptom Study

Forty-two (42) participants completing a weekly 51-point symptom questionnaire while using neurotransmitter precursors according to the present invention. The active ingredients were identical in type, dose and delivery to those in Example 1. The treatment program lasted five weeks. At the start of the program and each week thereafter, the patients filled out a 51-point questionnaire regarding known symptoms of neurotransmitter deficiencies. These include symptoms relating to their appetite, cravings, snacking, energy, and mood. Initially, some patients reported an increase of symptoms as they became more aware of them.

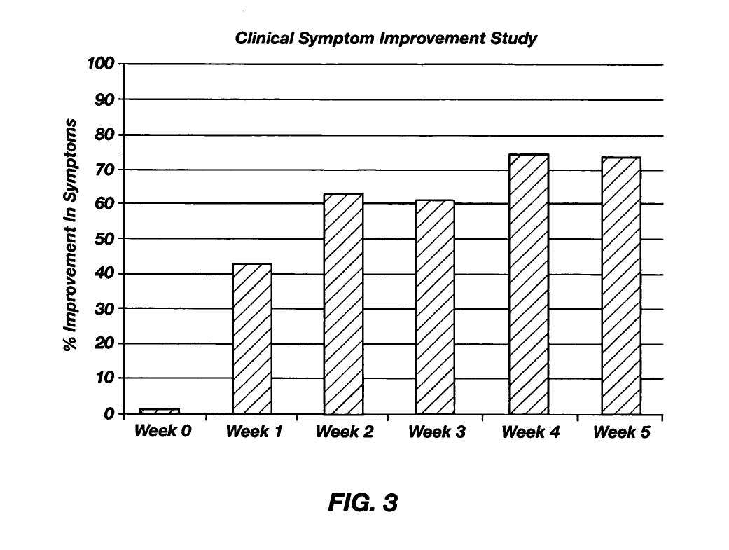

After four weeks, the average of all patient reports resulted in a decline of 76% of their initial symptoms (FIG. 3). Ninety percent of all patients reported a decline in at least half of their initial symptoms. Ten patients (24%) reported a complete relief (100% decline) of all 51 possible symptoms of neurotransmitter deficiencies.

### Example 6

Double-Blind Symptom Improvement Study

The study was done to see if taking dietary supplements prepared according to the Example 1 as directed could alleviate the known symptoms of neurotransmitter deficiencies. Since it has been determined (Example 4) that taking such dietary supplements increase serotonin and dopamine levels, it was hypothesized that the use of this product would also alleviate the symptoms of deficiencies when the participants increased their internal production of serotonin and dopamine.

Sixteen women volunteers were randomly divided into two equal groups—active and placebo. Each group contained 8 women at the beginning of the study. One of the women in the “active” group did not complete the study. The active group participants were given the dietary supplement product of Example 1, while the placebo group was given the same product without active ingredients. The participants were provided with enough product to take 2 lozenges, three times per day—6 total per day. They were instructed to dissolve the lozenges in their mouths and not chew them.

The participants filled out symptom rating responses on a weekly basis. Each of 23 symptoms of neurotransmitter deficiencies was listed in a chart. These were: depressed mood, fatigue, low motivation, poor focus, poor muscle strength or feeling weak, anxiety or worry, fearfulness, PMS-related moodiness, irritability, anger, chronic pain, achy muscles, sleep problems, cravings in the afternoon or evening, eating large food portions, feeling not satisfied after eating, thinking about food often, crave chocolate, crave caffeine, crave nicotine, crave starchy foods, crave sweets, and crave alcohol. The participants rated how strongly each symptom applied to them on a zero to five point scale, a “5” being the strongest, and “0” being non-existent.

The percent change of each symptom during the six-week period for each participant was determined. Then, the average percent change of each symptom for the entire group was calculated. A decrease in the severity of a symptom was termed an “improvement.” The percent improvement is based on the lessening of the severity as compared to the initial rating. For instance, if a symptom was rated at a “3” at the beginning of the study and as at a “1” at the end, then the symptom has declined by ⅔ or 66.7%. Similarly if a symptom goes from a “4” to a “3” it has declined (or improved) by ¼ or 25%. Whenever an existing symptom (i.e. the initial rating was “1” or more) goes to a rating of “0”, it has decreased (improved) by 100%.

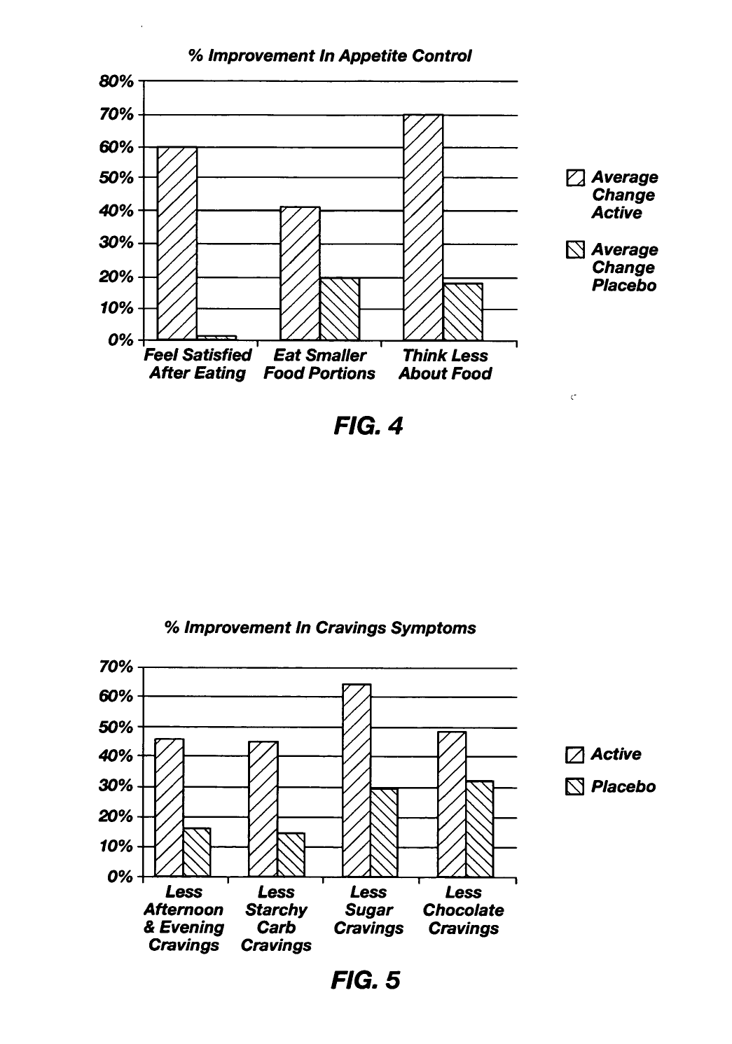

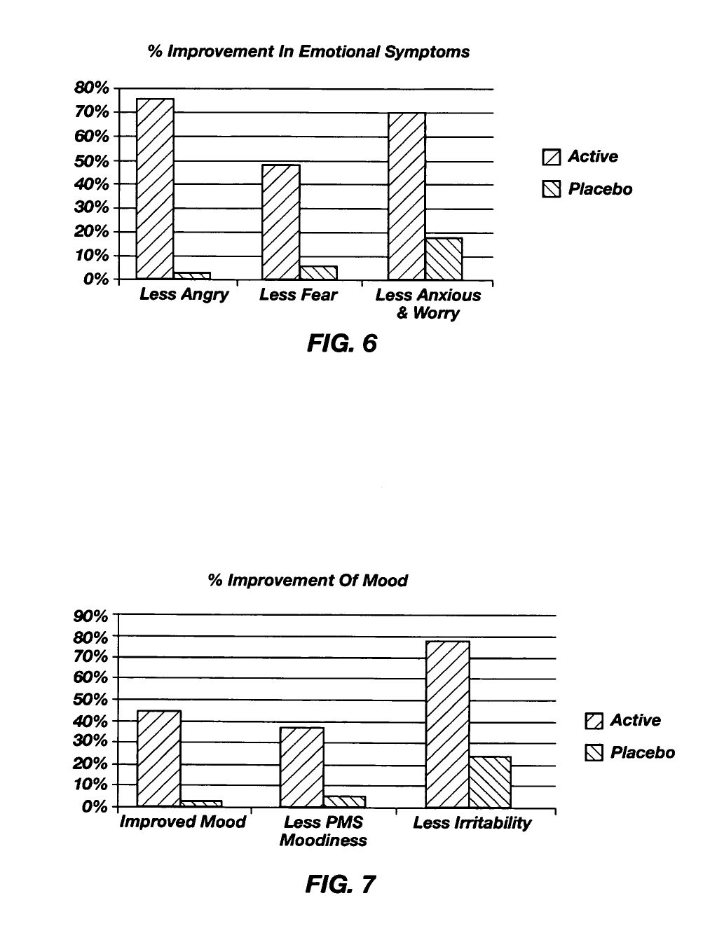

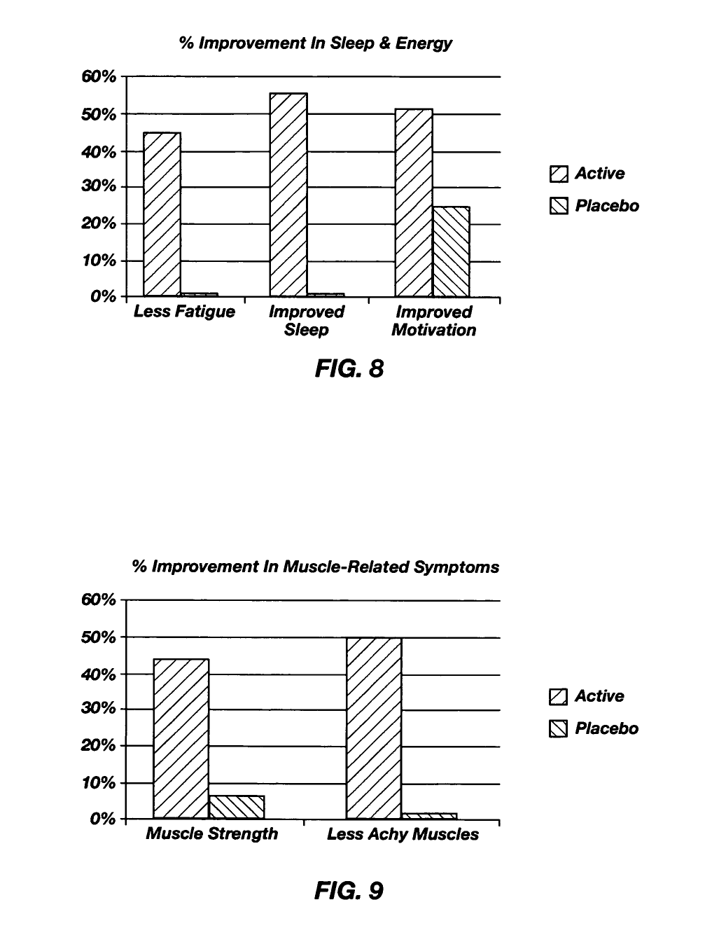

The results from the seven participants in the active group who completed the study showed a decrease in the severity of 22 out of the 23 symptoms of neurotransmitter deficiencies compared to the results of the placebo group. However, the decreases in three of the symptoms were not significantly greater than those of the placebo group. Significant differences in symptom ratings occurred in 19 of the 23 symptoms. The average improvement over all of the 23 symptoms was 3.5 times greater (+350%) in the active group compared to the placebo group. The top seven most affected symptoms were improved 55 times more (5500%) in the active group than in the placebo group. FIGS. 4-9 summarize the results for appetite control (FIG. 4), cravings (FIG. 5), emotional symptoms (FIG. 6), mood (FIG. 7), sleep and energy (FIG. 8), and muscle-related symptoms (FIG. 9).

In the 19 symptoms that had significant improvement, the average improvement of the active group was 59%, with the range from 30% to 81%. In these same 19 symptoms, the placebo group averaged 18% improvement with a range of 0 to 34%.

The most meaningful results relate to each symptom separately. There were 7 symptoms in which the improvement of the active group was more than ten times (>1000%) that of the placebo group. These were: depressed mood, fatigue, anxiety or worry, achy muscles, sleep problems, and not feeling satisfied after eating (eating satiation).

The active group had significantly more improvement than the placebo group in the symptoms of fearfulness (500%), PMS moodiness (440%), muscle strength (460%), thinking a lot about food (260%), irritability (210%), focus (190%), craving starchy carbohydrates (190%), cravings in afternoon or evening (170%), craving sweets (110%), motivation (100%), food portions (100%), and chocolate cravings (50%).

Therefore, the use of dietary supplements according to the present invention as directed over a six-week period affects the symptoms of neurotransmitter deficiencies in the following ways: improves eating satisfaction, improves mood, lowers anger and irritability, lessens fatigue, lowers anxiety and fearfulness, relieves achy muscles, improves sleep, lessens appetite and cravings especially for starchy or sweet carbohydrates; and improves motivation, mental focus and muscle strength.

1. A dietary supplement composition comprising a mixture of a first precursor of a first neurotransmitter and a cofactor for activating in vivo enzymatic synthesis of the first neurotransmitter from the first precursor or of a second neurotransmitter.

2. The dietary supplement composition of claim 1 wherein the first neurotransmitter comprises dopamine or norepinephrine.

3. The dietary supplement composition of claim 2 wherein the first precursor comprises a mixture of DOPA and tyrosine.

4. The dietary supplement composition of claim 2 wherein the first precursor comprises DOPA.

5. The dietary supplement composition of claim 2 wherein the first precursor comprises tyrosine.

6. The dietary supplement composition of claim 1 wherein the cofactor comprises vitamin B-6.

7. The dietary supplement composition of claim 1 further comprising a second precursor of the second neurotransmitter.

8. The dietary supplement composition of claim 6 wherein the second neurotransmitter comprises serotonin.

9. The dietary supplement composition of claim 8 wherein the second precursor comprises 5-hydroxytryptophan.

10. The dietary supplement composition of claim 9 further comprising a third neurotransmitter.

11. The dietary supplement composition of claim 10 wherein the third neurotransmitter comprises glutamine.

12. The dietary supplement composition of claim 1 further comprising a third neurotransmitter.

13. The dietary supplement composition of claim 12 wherein the third neurotransmitter comprises glutamine.

14. The dietary supplement composition of claim 1 further comprising a low-digestible carbohydrate sweetener.

15. The dietary supplement composition of claim 14 wherein the low-digestible carbohydrate sweetener is a member selected from the group consisting of isomalt, maltitol, and mixtures thereof.

16. The dietary supplement composition of claim 14 further comprising a non-nutritive sweetener.

17. The dietary supplement composition of claim 16 wherein the non-nutritive sweetener is a member selected from the group consisting of acesulfame-K, sucralose, and mixtures thereof.

18. The dietary supplement composition of claim 1 wherein the cofactor comprises vitamin B-6, the first precursor comprises 5-hydroxytryptophan, and the first neurotransmitter comprises serotonin.

19. A dietary supplement composition comprising a mixture of a first precursor of a first neurotransmitter and a cofactor for activating in vivo enzymatic synthesis of a second neurotransmitter.

20. The dietary supplement composition of claim 19 wherein the first neurotransmitter comprises serotonin.

21. The dietary supplement composition of claim 20 wherein the first precursor comprises 5-hydroxytryptophan.

22. The dietary supplement composition of claim 19 wherein the second neurotransmitter comprises dopamine or norepinephrine.

23. The dietary supplement composition of claim 22 wherein the cofactor comprises vitamin B-6.

24. The dietary supplement composition of claim 19 further comprising a low-digestible carbohydrate sweetener.

25. The dietary supplement composition of claim 24 wherein the low-digestible carbohydrate sweetener is a member selected from the group consisting of isomalt, maltitol, and mixtures thereof.

26. The dietary supplement composition of claim 24 further comprising a non-nutritive sweetener.

27. The dietary supplement composition of claim 26 wherein the non-nutritive sweetener is a member selected from the group consisting of acesulfame-K, sucralose, and mixtures thereof.

28. A dietary supplement composition comprising a mixture of:
(a) a base comprising a low-digestible carbohydrate sweetener;
(b) a cofactor for activating in vivo enzymatic synthesis of a first neurotransmitter; and
(c) a precursor of the first neurotransmitter or of a second neurotransmitter.

29. The dietary supplement composition of claim 28 wherein the cofactor comprises a vitamin.

30. The dietary supplement composition of claim 29 wherein the vitamin comprises vitamin B-6.

31. The dietary supplement composition of claim 28 wherein the low-digestible carbohydrate sweetener comprises isomalt, maltitol, or mixtures thereof.

32. The dietary supplement composition of claim 28 further comprising a non-nutritive sweetener.

33. The dietary supplement composition of claim 32 wherein the non-nutritive sweetener comprises acesulfame-K, sucralose, or mixtures thereof.

34. The dietary supplement composition of claim 28 further comprising a third neurotransmitter.

35. The dietary supplement composition of claim 34 wherein the third neurotransmitter comprises an amino acid.

36. The dietary supplement composition of claim 35 wherein the amino acid comprises glutamine.

37. The dietary supplement composition of claim 28 wherein the precursor comprises DOPA, tyrosine, 5-hydroxytryptophan, or mixtures thereof.

38. A method of treating a neurotransmitter deficiency comprising orally administering an effective amount of a dietary supplement composition comprising a mixture of a first precursor of a first neurotransmitter and a cofactor for activating in vivo enzymatic synthesis of the first neurotransmitter from the first precursor or of a second neurotransmitter.

39. The method of claim 38 wherein the dietary supplement composition is administered such that the first precursor and the cofactor are absorbed through the mucosa of the oral cavity.
